(19) United States
(12) Patent Application Publication
(10) Pub. No.: US 2010/0168219 A1
(43) Pub. Date: Jul. 1, 2010

(54) Chronic inflammation and transplantation

(76) Inventor: Jonathan Steven Alexander, Shreveport, LA (US)

Correspondence Address: Dr. Trevor P. Castor;CEO, Aphios Corporation, 3-E Gill Street, Woburn, MA, 01801, US

(21) Appl. No.: 12/319,004

(22) Filed: Dec. 31, 2008

(51) Int. Cl. A61K 31/365; A61P 29/00

(52) U.S. Cl. 514/453

(57) ABSTRACT

Neutrophils (PMN) can migrate along gradients of chemoattractants across endothelial monolayers to sites of inflammation and infection. This chemotaxis through endothelial cell borders is involved in several acute and chronic inflammatory diseases, however our understanding of the role of endothelial second messengers in the regulation of leukocyte emigration is still incomplete. We investigated this using an in vitro model of neutrophil migration across human umbilical vein endothelial cells (HUVECs) and human microvascular endothelial cells (HMECs) on cell culture inserts. We report that activation of endothelial protein kinase C (PKC) by both phorbol myristate acetate (PMA) and Bryostatin-1 (a potent PKCδ and c activator) can completely abolish neutrophil migration mediated by both endothelial TNF-α stimulation and a leukotriene B4 (LTB₄) gradient. PMA protected against LTB₄ induced PMN transmigration for at least 24 hours in HMECs and HUVECs. Bryostatin-1 protected PMN migration for at least 24 hours in HMECs and at least 48 hours in HUVECs. Pretreatment with Go-6983 (PKCα, β, and δ inhibitor) before the addition of Bryostatin-1 restored the loss of LTB₄ induced neutrophil migration, while pretreatment with GO-6976 (PKCα and β inhibitor) did not. In addition using PKCδ and ε specific small interfering RNA, we were able to show that PKCδ, but not ε was at least mostly responsible for the loss of neutrophil migration in response to LTB₄. Taken together, these observations suggest that activation of endothelial PKCδ could be therapeutic in the treatment of various inflammatory disorders characterized by enhanced neutrophil infiltration.

This invention relates to pharmaceutical compositions, particularly pharmaceutical compositions comprising bryostatin-1 and substituted derivatives of bryostatin-1, thereof as pharmaceuticals for inhibition of inflammation, and for use in combating arteriosclerosis, diseases of the cardiovascular system, of the central nervous system and prior to/following organ transplantation, ischemia. The invention relates to methods for treating leukocyte dependent injury in chronic inflammatory diseases, and injury from transplantation mediated organ stress. The method involves injecting bryostatin-1 into patients with the inflammatory condition, treating the skin with bryostatin-1, or perfusing organs with bryostatin-1 prior to transplantation/cold storage. Activation of protein kinase Cd (PKCd) results in a near complete blockade of leukocyte infiltration which is the result of stabilization of the microvascular (endothelial) barrier.

### GOVERNMENT SUPPORT

Research leading to this invention was in part funded with Grant No. NIH DK-43785 from the National Institutes of Health, Bethesda, Md., USA.

### FIELD OF THE INVENTION

The invention relates to methods for treating leukocyte dependent injury in chronic inflammatory diseases, and injury from transplantation mediated organ stress. The method involves injecting bryostatin-1 into patients with the inflammatory condition, treating the skin with bryostatin-1, or perfusing organs with bryostatin-1 prior to transplantation/cold storage. Activation of protein kinase Cd (PKCd) results in a near complete blockade of leukocyte infiltration which is the result of stabilization of the microvascular (endothelial) barrier. This invention relates to pharmaceutical compositions, particularly pharmaceutical compositions comprising a bryostatin-1, other bryostatins and substituted derivatives of bryostatins for use in treating inflammation, and for use in combating arteriosclerosis, diseases of the cardiovascular system, of the central nervous system and prior to/following organ transplantation, ischemia.

### BACKGROUND OF THE INVENTION

Neutrophil mediated tissue injury is an extremely important aspect of both acute and chronic inflammatory disease processes. Methods to reduce neutrophil infiltration could provide important therapies in numerous diseases including stroke, inflammatory bowel disease, arthritis and atherosclerosis. Generally the vascular endothelium exists in a quiescent state, without rolling, adhering, or transmigrating leukocytes. In vitro there is consistent and reproducible evidence suggesting that an almost insignificant amount of neutrophil transendothelial migration takes play through an unstimulated endothelial monolayer. However, in the presence of an exogenously applied chemoattractant, or upon endothelial activation (e.g. with Th1 cytokines, LPS), significant quantities of neutrophils will migrate through endothelial monolayers.

Leukocyte transmigration is usually referred to as a three step process involving: 1) rolling along the endothelium 2) firm adhesion to the endothelium and lastly 3) migration across the endothelial monolayer into underlying tissues. The process of transendothelial migration begins when the leukocytes first undergo selectin-mediated rolling followed by integrin-mediated firm adhesion. The first two steps are mechanistically well-characterized and result in the accumulation of leukocytes on the luminal surface of vascular endothelial cells. Importantly, leukocyte-endothelial signaling through adhesion molecules and integrins during these steps is crucial for the final stage of transendothelial migration or diapedesis involving migration of the neutrophil in an amoeboid manner through the monolayer followed by leukocyte migration within the interstitium. The current general paradigm suggests that neutrophil migration occurs through endothelial cell junctions and is aided through endothelial cell adhesion molecule interactions including ICAM-1, PECAM-1, CD99, LSP-1, and IAP. However, the role of endothelial second messengers and their possible regulation of neutrophil migration have not been investigated as extensively.

During inflammation we recognize at least two distinct forms of leukocyte migration: acute, chemoattractant mediated transmigration (Type I) and chronic or cytokine-activated transmigration (Type II). Type I requires only a chemotactic gradient of factors such as leukotriene B4 (LTB₄), platelet activating factor (PAF), or n-formyl-met-leu-phe (fMLP), and has reported to be PECAM-1 independent (an adhesion molecule suggested to mediate leukocyte transendothelial migration), despite the presence of PECAM-1 on both neutrophils and endothelial cells. This type of migration usually requires only minutes to activate, lasts for hours and does not involve protein synthesis. In contrast, type II migration is ‘chronic’ and requires prior ‘activation’ of the endothelium. Type II migration relies on transcription/translation of molecules including endothelial cell adhesion molecules (and possibly several other inflammatory mediators and chemoattractants e.g. chemokines). While Type II migration can almost totally be abolished by PECAM blockade, PECAM appears to play no role in Type I TEM. Studies performed in vitro and in vivo characterizing the involvement of both endothelial cell-cell junctions and endothelial cell adhesion molecules in PMN transmigration are discussed in several reviews.

While junctional alterations caused by neutrophils may lead to enhanced endothelial permeability, neutrophil extravasation and vascular permeability regulation are independently controlled. Interestingly though, there is some overlap in factors mediating permeability (histamine, thrombin) and endothelial signals produced by neutrophil adhesion such as Ca⁺⁺, MAPK, ERK, and myosin light chain kinase. Due to the fact that increased vascular endothelial cell permeability and gap formations through which neutrophils pass are often assumed to be distinct features of acute inflammation, numerous researchers have investigated the role of these second messengers and the function of the endothelial cell contractile apparatus in the modulation of neutrophil migration.

Numerous signals are initiated upon neutrophil adherence to the endothelial monolayer that has been suggested to direct changes in the contractile state of the cell and result in junctional alteration increasing PMN TEM. One of the first is a transient increase in intracellular cytosolic free calcium. The next step in this process has been described to be the activation of myosin light chain kinase leading to phosphorylation of myosin light chains resulting in increased isometric tension. The final effect is a relaxation of endothelial junctional contacts, endothelial retraction, and the formation of subsequent space facilitating neutrophil passage.

These observations have led to studies on the outcomes of inhibiting these second messengers on neutrophil migration in response to various substances. It has been reported that endothelial cytoskeletal alterations could change PMN TEM in response to fMLP. This was further supported by studies using the blockade of intracellular calcium release and myosin light chain kinase in the endothelium to inhibit neutrophil migration in response to LTB₄ gradients. Enhanced intracellular Ca⁺⁺ has been suggested to be essential for myosin light chain phosphorylation and thus gap formation between endothelial cells. In addition to Ca⁺⁺, Rho kinase blockade upstream of MLC phosphorylation as well as actin polymerization has been implicated in inhibition of neutrophil TEM in response to LTB₄ through interactions with MLC phosphatase. Moreover mitogen-activated protein kinase (MAPK) and extracellular signal-regulated kinase (ERK) activation have also been suggested to regulate neutrophil TEM in response to both an fMLP gradient and activation of the endothelium by TNF-α.

Consequently, some palpable endothelial second messengers have previously been investigated in the control of neutrophil migration. We therefore set out to define other less evident second messengers that could alter neutrophil TEM in response to LTB₄ and TNF-α. We screened activation and inhibition of a variety of endothelial second messengers and found the most dramatic effects with PKC activators. Our studies suggest activation of specifically PKCδ, but not PKCδ can inhibit neutrophil transendothelial migration in response to both LTB₄ and TNF-α. In addition, our studies suggest that short term activation of PKCδ can inhibit PMN TEM long-term, up to 48 hours, without affecting endothelial viability. We therefore suggest PKCδ activation may be therapeutically beneficial in numerous inflammatory disorders.

### SUMMARY OF INVENTION

The method for treating inflammatory disease and transplantation according to the invention is characterized in that it includes: (i) the incubation of organs with one or more bryostatin-1 derivatives under conditions which allow vascular exposure to these compounds prior to or immediately following ‘harvesting’, and (ii) intravenous, transdermal, intraperitoneal, intralveolar instillation of bryostatin-1 in vivo during active disease, or during periods of disease remission.

### BRIEF DESCRIPTION OF THE DRAWINGS

FIG. 1: Neutrophil adhesion to and migration through HMECs upon stimulation with an LTB₄ gradient over time. HMECs were plated on 8 μm transwell inserts and allowed to reach confluency for migration assays. For the adhesion assay, HMECs were plated on 48 well plates. LTB₄ and 500,000 neutrophils were added to the upper compartment to initiate adhesion, while 500,000 neutrophils were added to the upper compartment and LTB₄ to the lower compartment to initiate migration. Both neutrophil adhesion and migration were measured over 3 hrs and expressed as % change in adhesion and migration vs. control. An inverse relationship was observed between adhesion and migration with maximal significant adhesion observed at 1 hr (p<0.001) and maximal significant migration observed by 2 hr (p<0.001).

FIG. 2: Dose dependent response of PMA on neutrophil migration. The chemotactic agent 100 nM LTB₄ significantly increased neutrophil migration (***=p<0.001) across confluent HMECs grown on transwells compared to control after 3 hr incubation. Pretreatment with 100 nM, 10 nM, and 1 μM PMA (1 hr) reduced LTB₄ induced migration in a dose dependent manner (***=p<0.001). Lower concentrations of PMA (1 nM) have no affect on LTB₄ induced migration.

FIG. 3: Dose dependent response of Bryostatin-1 on neutrophil migration. The chemotactic agent 100 nM LTB₄ significantly increased neutrophil migration (***=p<0.001) across confluent HMECs grown on transwells compared to control after 3 hr incubation. Pretreatment with 100 nM and 10 nM Bryostatin-1 (1 hr) reduced LTB₄ induced migration in a dose dependent manner (***=p<0.001). Lower concentrations of Bryostatin-1 (1 nM) have no affect on LTB₄ induced migration.

FIG. 4: Effect of PMA on TNF-α induced neutrophil migration. Endothelial activation by 24 hr pretreatment with 10 or 20 ng/ml TNF-α significantly increased neutrophil migration (P<0.05) across confluent HMEC monolayers grown on transwells. This TNF-α induced TEM was totally attenuated by 1 hour pretreatment with 100 nM PMA (p<0.001).

FIG. 5: Effect of Bryostatin-1 on TNF-α induced neutrophil migration. Endothelial activation by 24 hr pretreatment with 10 or 20 ng/ml TNF-α significantly increased neutrophil migration (P<0.05) across confluent HMEC monolayers grown on transwells. This TNF-α induced TEM was totally attenuated by 1 hour pretreatment with 100 nM Bryostatin-1 (p<0.001).

FIG. 6: Protection from LTB₄ induced PMN TEM with PMA up to 48 hr after pretreatment. HMECs were pretreated for 1 hr. with PMA and washed with HBSS three times 0, 24, or 48 hours before migration was initiated. Migration was stimulated by adding 500,000 neutrophils to the top well and 100 nM LTB₄ to the lower well at 0, 24, and 48 hours after PMA pretreatment. The chemotactic agent LTB₄ significantly increased neutrophil migration (p<0.001) compared to control after 3 hr incubation. PMA (100 nM) reduced LTB₄ induced migration up to 48 hours after 1 hr endothelial pretreatment (p<0.001).

FIG. 7: Protection from LTB₄ induced PMN TEM with Bryostatin-1 up to 48 hr after pretreatment. HMECs were pretreated for 1 hr. with Bryostatin-1 and washed with HBSS three times 0, 24, or 48 hours before migration was initiated. Migration was stimulated by adding 500,000 neutrophils to the top well and 100 nM LTB₄ to the lower well at 0, 24, and 48 hours after Bryostatin-1 pretreatment. The chemotactic agent LTB₄ significantly increased neutrophil migration (p<0.001) compared to control after 3 hr incubation. Bryostatin-1 (100 nM) reduced LTB₄ induced migration up to 48 hours after 1 hr endothelial pretreatment (p<0.001).

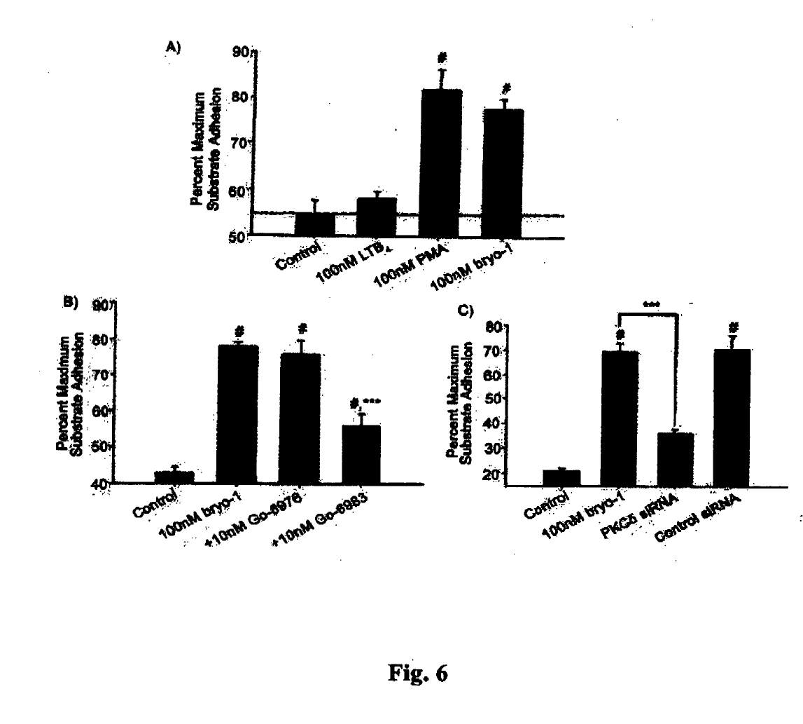

FIG. 6: Protection from LTB₄ induced PMN TEM with PMA up to 48 hr after pretreatment.

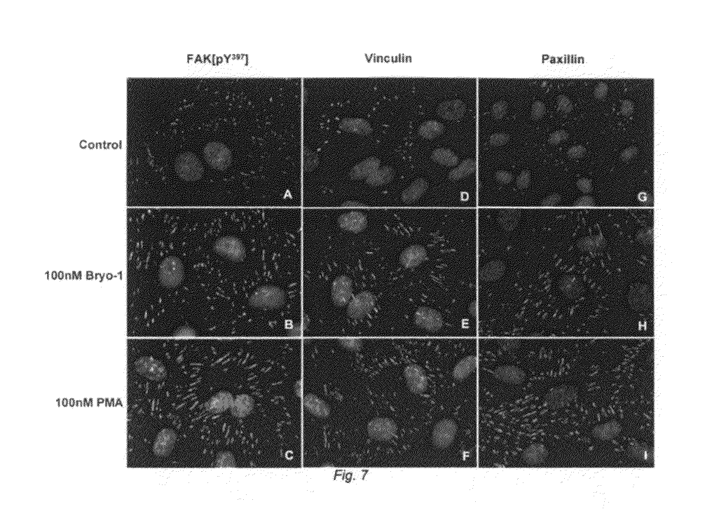

FIG. 7: Protection from LTB₄ induced PMN TEM with Bryostatin-1 up to 48 hr after pretreatment.

### DETAILED DESCRIPTION OF THE INVENTION

Reagents and Abs: LTB₄, PMA, and Bryostatin-1 were purchased from Biomol (Plymouth, Pa.). Alamar blue solution was from Biosource International (Camarillo, Calif.). PKCε siRNA SMARTpool, non-specific control siRNA pool, and anti-PKCε monoclonal antibodies were obtained from Upstate Cell Signaling (Lake Placid, N.Y.). PKCd siRNA (Duplex 2) was purchased from Molecular (Sterling, Va.) and the anti-PKCδ polyclonal antibody was from Chemicon (Temecular, Calif.). Nucleofection reagents were obtained from Amaxa (Gaithersburg, Md.).

Subjects: The procedure used to obtain human neutrophils was approved by the Institutional Review Board for Human Research at the Louisiana State University Health Sciences Center. Each subject provided written consent for participation in the study.

Cell Culture: HMECs were a generous gift from Dr. Francisco Candal (Centers for Disease Control), and are derived from the subcutaneous microvasculature. HMECs were maintained in MCDB-131 supplemented with 10% FCS, 1% antibiotic antimycotic, 10 ng/ml EGF, and 1 μg/ml hydrocortisone. The cell cultures were incubated at 37° C. in a humidified atmosphere with 7.5% CO₂ and expanded by brief trypsinization (0.25% trypsin in phosphate-buffered saline containing 0.02% EDTA). HMECs were seeded onto 8 μm inserts for migration assays and 48 well plates for endothelial viability studies. Human umbilical vein endothelial cells (HUVECs) were harvested from umbilical cords by 0.25% collagenase treatment for 20 min at 37° C. HUVECs were maintained in Endothelial Growth Medium (EGM) supplemented with Bovine Brain Extract. The cell cultures were incubated at 37° C. in a 100% humidified atmosphere with 5% CO₂ and expanded by brief trypsinization. Primary passage HUVEC were seeded onto fibronectin coated (25 μg/ml) 8 μm inserts for migration assays and 48 well plates for endothelial viability studies. Culture medium was replaced every second day. Cells were identified as endothelial cells by their cobblestone appearance at confluency, positive labeling with acetylated low density lipoprotein labeled with 1,11-dioctadecyl-1 3,3,31,31,3-tetramethylindocarbocyanine perchlorate (Dil-Ac-LDL; Biomedical Technologies, Inc.) and mouse antihuman factor VIII (Calbiochem, San Diego, Calif.).

Neutrophil Isolation: Neutrophils were isolated using a Ficol gradient. Whole blood was taken from human donors, and 5 ml was layered on top of two layers of Histopaque. Tubes were spun at 2100 RPM for 40 min. The neutrophil layer was added to cold PBS and respun at 2100 RPM for 5 min. Supernatant was removed and neutrophils were suspended in a fixed amount of PBS to determine cell count. Cells were kept on ice until assays were performed.

Migration Assays: At confluency HMECs were treated for 1 hr at 37° C. with Bryostatin-1 (10 nM, 100 nM) in MCDB-131, or MCDB-131 alone. Inserts were washed three times with HBSS (to eliminate drug effects on the neutrophils) and placed in a new plate containing either 500 μL 100 nM LTB₄, or HBSS alone for control. 500 μL HBSS was then added to the top well and spiked with 500,000 neutrophils. For TNF-α experiments, HMECs were left in 20 ng/ml TNF-α in MCDB-131 for 24 hr. TNF-α was removed and HMECs washed three times. HMECs were treated for 1 hr with Bryostatin-1 in EGM (10 nM, 100 nM). The drug was removed and cells were washed three times. 500 μL HBSS was added to both upper and lower chambers and spiked with 500,000 neutrophils. Neutrophils were allowed to migrate for 3 hr at 37° C. Migration was stopped by removing the inserts and migration was measured using the MPO assay.

Time Course Assays: At confluency, HUVECs were treated for 1 hr at 37° C. with Bryostatin-1 (10 nM, 100 nM) in EGM or EGM alone. Inserts were washed three times with HBSS (to reduce drug effects on the neutrophils) and migration assays were ran at t=0, 24, or 48 hr. After time elapse HUVECs placed in a new plate containing either 500 μL 100 nM LTB₄, or HBSS alone for control. 500 μL HBSS was then added to the top well and spiked with 500,000 neutrophils. Neutrophils were allowed to migrate for 3 hr at 37° C. Migration was stopped by removing the inserts and migration was measured using the MPO assay.

Neutrophil TEM Restoration Studies: HUVECs were pretreated with Go-6983 (1, 10, 100 nM), or Go-6976 (1, 10, 100 nM) for 30 minutes, then spiked with 100 nM Bryostatin-1 for 1 hour. HUVECs previously nucleofected with PKCδ, ε, or control siRNA were just treated in Bryostatin-1 for 1 hour. HUVECs were washed three times and placed in a new plate containing either 500 μL 100 nM LTB₄, or HBSS alone for control. Neutrophils were allowed to migrate for 3 hr at 37° C. Migration was stopped by removing the inserts and migration was measured using the MPO assay.

Myeloperoxidase Assay: Neutrophil migration was measured using myeloperoxidase (MPO) analysis. Cell culture inserts were first removed and plates were spun at 1500 RPM for 5 min. The supernatant was removed, and MPO activity was measured using TMB/peroxide as chromogen. The reaction was stopped with sulfuric acid and absorbance was read at 450 nm using a microplate reader. All experiments were performed n=6. Data was expressed as “% neutrophil migration”, which allowed cumulative statistical analysis to be done and account for unpreventable batch-to-batch variations.

Viability Assay. The Alamar blue assay was used to determine endothelial viability after pharmacological intervention. Alamar blue contains a REDOX indicator that results in a color change upon chemical reduction by cellular metabolic activity. Endothelial cells were treated with bryostatin-1 (100 nM) for 1 hr and washed three times with HBSS. Alamar blue (10%) in media was added for 2.5 hr at T=0, 24, or 48 hr after drug treatment and the ratio of absorbances (570-600 nm) measured to determine metabolic activity.

siRNA. Optimum nucleofection parameters were determined using nucleofection of a control vector pmaxGFP. Using fluorescence microscopy and flow cytometry 2 μg vector/500,000 cells nucleofected yielded the highest nucleofection (data not shown). HUVECs were grown to 70% confluency, trypsinized, and then counted. 500,000 cells were spun down 5 min at 1500 RPM and resuspended in 100 μL HUVEC nucleofector solution and 2 μg PKCδ, PKCε, or control siRNA. Suspensions were transferred to an Amaxa certified cuvette and nucleofected using a Nucleofector Device that uses a unique combination of electrical parameters to deliver the siRNA directly to the cell nucleus. 500,000 nucleofected HUVECs were then transferred either to one fibronectin coated 8 μm insert, or one fibronectin coated well in a 12 well plate. Migration assays and Western blotting was performed 24 hours after nucleofection for optimal knockdown.

Statistical Analysis: All values are expressed as means+SE. Data were analyzed using a one-way ANOVA with Bonferroni corrections for multiple comparisons. Probability (P) values of <0.05 were considered significant.

Dosage form: is a means for administering a drug. An oral dosage is a tablet, capsule, powder, or liquid for ingestion. An oral solid dosage form in a tablet, capsule or powder.

Pharmaceutical parenteral formulation: a sterile, isotonically acceptable and pH acceptable, aqueous solution or suspension of a drug for direct injection into the body or for perfusing one or more organs.

Pharmaceutically acceptable salt: a drug that has been modified to present a salt of physiologically acceptable anion or cation.

Bryostatin-1 analog: shall mean a composition having the general formula of bryostatin-1 with substitutions comprising methyl or ethyl groups or halogens and ammonium groups which do not substantially alter the biological activity of the composition.

These studies clearly identify a role for a previously unreported second messenger in the prevention of both Type I and Type II neutrophil infiltration. We have clearly shown that PKC activation, more specifically PKCδ activation can totally abolish neutrophil TEM in response to an LTB₄ gradient, or across TNF-α activated endothelial cells. In addition, we show that a short term activation of PKC can protect against induced neutrophil infiltration up to 48 hours after the initial drug treatment.

We used a variety of pharmacological activators and inhibitors to determine second messengers that could be used to inhibit reduce neutrophil infiltration in an in vitro model of inflammation. We tested some pharmacological inhibitors that have previously been examined in this model including some that inhibit MLCK, ERK, MAPK, [Ca⁺⁺]ᵢ release, and that activate PKA through cAMP. In agreement with Huang et al. inhibition of endothelial intracellular Ca⁺⁺ release did inhibit PMN TEM in response to LTB₄. In addition, our data also further support a previous finding of a role for PKA activation in the reduction of neutrophil TEM in our model of human microvascular endothelial cells. However, our data disagreed with the findings of Garcia et al. and Stein et al. suggesting that MLCK and ERK1/2 inhibition, respectively, could limit leukocyte extravasation toward a chemoattractant gradient of LTB₄. While our data disagree with these findings, it could be due to variations in cell type and stimulus for the induction of neutrophil TEM. Both Garcia et al. and Stein et al. used HUVECs for their studies, and Stein et al. used fMLP as the stimulator for leukocyte extravasation.

More importantly, we found a total abolition of neutrophil transendothelial migration across HUVECs and HMECs towards both an LTB₄ gradient and endothelial TNF-α stimulation using two different PKC activators. These levels of reduction were similar only to those reported here for the blockade of intracellular Ca⁺⁺ release, and previously exhibited by Huang et al. This is interesting, because Ca⁺⁺ has been suggested by numerous investigators to be the first second messenger increased upon neutrophil adhesion to an endothelial monolayer. While no studies have shown alterations in PKC activity upon neutrophil adhesion to an endothelial monolayer, endothelial PKC activation is just as potent as Ca⁺⁺ inhibition in reducing neutrophil TEM towards an LTB₄ gradient and in response to endothelial activation by TNF-α. It is possible that neutrophil adhesion to the endothelium does not result in any alterations in PKC activity, but it has been shown that activation of particular isoforms, including PKCα, β, γ, and δ can result in alterations in endothelial barrier function. This alteration could be involved in regulating neutrophil passage through the endothelial monolayer.

Isoforms of PKC are shown to be involved in numerous cellular processes ranging from apoptosis to cell proliferation and differentiation. There are three described PKC subgroups categorized by their structure, modes of activation, and their regulation including the conventional (α, βI βII, γ) novel (δ, ε, η, θ) and atypical (□ζ and ι/λ) isoforms. Activation of PKC has long been accepted as a mechanism responsible for reduced endothelial barrier integrity induced by numerous mediators including thrombin, VEGF, H₂O₂, glucose, and phorbol esters. While the exact mechanism of PKC mediated permeability has not been elucidated, PKC activation has been shown to target the endothelial cytoskeleton resulting in MLC phosphorylation and actin polymerization. In addition PKC activation has been shown to disassemble VE-cadherin, and induce reorganization of focal adhesions. A report from our own lab implicated a role for PKC in cadherin endocytosis and increased endothelial permeability. Many distinct isoforms of PKC have been implicated in the induction of endothelial permeability, though there seems to be no general consensus on the precise isoform.

The phorbol ester PMA (phorbol myristate acetate), a pan PKC activator, has been shown in numerous studies to decrease transendothelial resistance (TER) in several types of endothelial and epithelial cells. This is due to the activation and translocation of the PKCα isoform and not PKCδ or ε. Also, the treatment of porcine aortic endothelial cells with specific antisense oligodeoxynucleotides against PKCα reduces enhanced permeability induced by glucose. A role for the β isoform has also been implicated in increased permeability caused by high glucose and PMA. The PKCα inhibitor hypocrellin A and the PKCβ inhibitor LY379196 both reduced this increased permeability with a greater effect seen with PKCα inhibition. Numerous studies have supported a role for PKCα in thrombin and PMA-mediated permeability in HUVECs, TNF-a-induced permeability in pulmonary microvessel endothelial cells, and lysophosphatidylcholine (LPC)-stimulated permeability in human dermal endothelial cells highly implicating PKCα as an important mediator of endothelial barrier function.

PKCδ and η have been linked to hyperpermeability of pulmonary microvascular endothelial cells as a result of PMA treatment. It is important to note that these last findings are in pulmonary endothelia and may represent a unique mechanism to the rest of the vasculature. In fact, an over-expression of PKCα significantly elevated permeability in thrombin stimulated rat epididymal microvascular endothelial cells, while PKCδ over-expression significantly blunted thrombin-induced increases in permeability. This data further supports the role of PKCα in permeability, and suggests that PKCδ may have opposing effects on permeability depending on the vascular bed. This represents a possible mechanism for the reduction in neutrophil migration through enhancement of endothelial barrier function induced by activation of PKCδ.

In addition, PKCδ has been shown to modulate activation of NFκB, a transcription factor that plays a key role in regulating both immune and inflammatory responses. While PKCδ activation via thrombin has been shown to increase ICAM-1 expression, we have shown that Bryostatin-1 protects against LTB₄ induced migration for up to 48 hours and protects against TNF-α induced migration a factor known to also promote NFkB activity and ICAM expression. This suggests that even if Bryostatin-1 did promote NFkB activity through activation of PKCδ, it is still able to protect against neutrophil TEM through some other mechanism.

Because PMA stimulates such a variety of endothelial second messengers, we chose to primarily focus on the effects of Bryostatin-1 in reducing neutrophil TEM. In addition, the effects of PMA could not be reversed with any PKC inhibitors, and we therefore could not guarantee that the actions of PMA were directly a response to the activation of PKC. Bryostatin-1 is a macrocyclic lactone isolated from the marine invertebrate Bugula neritina that activates PKC in a unique way to phorbol esters. Importantly, it more potently activates the delta and epsilon isoforms. Bryostatin-1 is currently in Phase II trials used in combination with other drugs for the treatment of a variety of cancers. So far the only major side effects found to be associated with bryostatin-1 use are myalgias, nausea, and vomiting, but no cardiovascular disturbances or evidence of edema have been observed. It is important to note that continual bryostatin-1 treatment results in down-regulation in most PKC isoforms (α, β, ε), however, numerous studies have suggested that PKCδ is not down-regulated with continuous bryostatin-1 administration and can maintain activation at various concentrations. Bryostatin-1 (10-100 μM; 100 nM-1 μM) could block the down-regulation of PKCδ caused by PMA when co-applied. Therefore, due to maintenance of PKCδ activation, this agent could be translated to use for other disorders benefiting from consistent PKCδ activation including acute inflammatory states. This information is important due to the fact that PKCα has been suggested to decrease barrier function, while PKCδ has been shown to function inversely. Therefore, Bryostatin-1 may protect against inflammatory injury by down-regulating PKCα and activating PKCδ resulting in positive barrier effects.

Bryostatin-1 has been shown to both enhance barrier function and also inhibit the decrease in TER caused by TNF-α in T84 intestinal epithelia, as well as have no affect on T84 epithelial barrier integrity. However, there was a slight decrease in T84 epithelial TER, but it was only transient due to the fact that Bryostatin-1 rapidly down-regulates PKC-α. The fact that Bryostatin-1 has not been shown to negatively affect endothelial barrier suggests that either it does not activate isoforms that negatively regulate the barrier, or the isoforms that it does preferentially activate antagonizes these effects on barrier. To date however, there have been no published studies on the effects of Bryostatin-1 on endothelial barrier integrity.

Therefore, we have shown that PKCδ activation protects against LTB₄ induced neutrophil transendothelial migration in vitro.

According to the invention, in order to produce protection of tissues, the vasculature must be exposed to concentrations of bryostatin-1 ranging from 20-1000/ng (to achieve levels of 10⁷M). Results obtained with healthy subjects and cancer patients have shown no adverse effects to even higher dosing ranges.

The transplantation solution would similarly contain bryostatin-1 up to 10⁻⁷M, in which the transplantation solution is UW solution, Plegisol or other organ transplantation harvesting/storage buffer.

Bryostatin-1 represents bryostatin-1 or any compound which is based on the bryostatin structural backbone.

In a general manner, for the implementation of the method according to the invention, the minimal tissue incubation stage is carried out at ambient temperature, by perfusion loading the organ with a quantity of bryostatin-1, and according to the duration, allowing the desired interaction to be obtained.

In a tissue or systemic embodiment of the invention, bryostatin-1 is injected, inhaled, applied or aspirated to exposure select regions of the vasculature. In the case of dermal, peritoneal or alveolar application, concentrations in excess or below 10⁻⁷ M will be used depending on the condition being treated.

By way of example, bryostatin-1 can be mentioned as an activator of PKCδ.

The invention also relates to diagnostic kits or sets for the implementation of the test defined above. These kits are characterized in that they comprise, with instructions for use, bryostatin-1 as defined above, with, if appropriate, receptacles and reagents, these reagents being chosen from activators and/or inhibitors of PKC.

Therefore, the invention provides means of tissue protection which are potent, lasting and non-invasive, allowing the rapid and economical treatment of these diseases to be carried out.

### EXAMPLES

### Example 1

### Second Messenger Reduction of Neutrophil TEM in Response to LTB₄

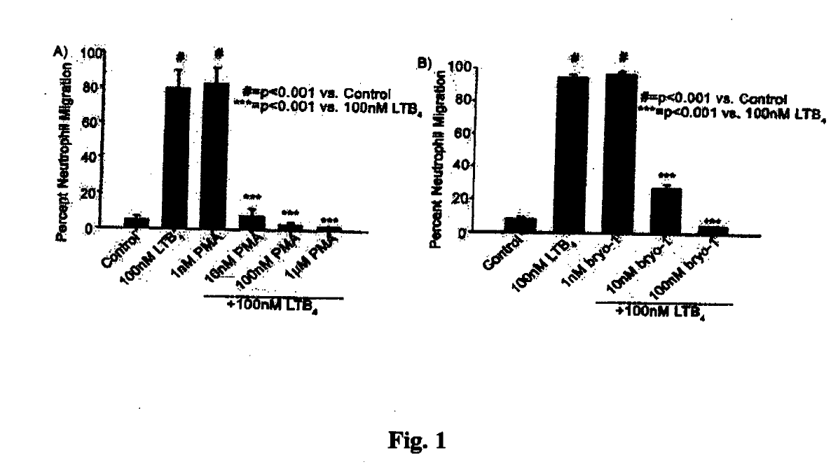

The time scale of neutrophil adhesion to and migration through a HMEC monolayer is represented in FIG. 1.

A 100 nM concentration of LTB₄ induces maximal adhesion at 1 hour accompanied by only a small increase in migration. As adhesion begins to decrease there is a corresponding increase in neutrophil migration. By 90 mins neutrophil migration is almost maximal and neutrophil adhesion has returned to baseline. To determine novel endothelial second messengers that could inhibit neutrophil transendothelial migration, endothelial cells were treated with a panel of pharmacological inhibitors and activators and alterations in neutrophil migration in response to LTB₄ were observed.

Table 1 lists the drugs used to screen alterations in neutrophil migration and the effects of each drug on neutrophil adhesion and migration.

TABLE 1
Second Messenger Alteration of Neutrophil Migration Induced by an LTB₄ Gradient.

| Drug | Concentration | | Effect on Migration | Function |
|---|---|---|---|---|
| A23187 | 10 | μM | No Effect | Ca⁺⁺ ionophore |
| AG-126 | 50 | μM | No Effect | Inhibits tyrosine kinases |
| Anisomycin | 10 | μM | Increased 14.61% ± 2.31% | Jun n-terminal kinase activator |
| Bryostatin-1 | 10 | nM | Decreased 103.8% ± 0.35% | PKCδ and ε activator |
| Dibutyryl cAMP | 1 | mM | Decreased 19.27% ± 1.91% | cAMP analog; activates cAMP-dependent protein kinase |
| Calyculin A | 1 | nM | No Effect | Inhibits protein phosphatase 1 and 2A |
| Calmodulin antagonist | 50 | nM | No Effect | Calmodulin antagonist |
| Ceramide C6 | 1 | μM | Decreased 45.44% ± 2.98% | Stimulates tyrosine phosphatases; activates MAP kinase |
| Chelerythrine Cl | 1 | μM | No Effect | Protein kinase C (PKC) inhibitor |
| Chloroquine | 80 | μM | No Effect | Blocks endocytosis |
| Cytochalasin D | 10 | nM | Decreased 32.97% ± 3.52% | Disrupts actin filaments |
| diphenyliodonium | 300 | nM | No Effect | Inhibits NADPH-utilizing flavoproteins |
| Dibutyryl cGMP | 1 | mM | No Effect | Activates cGMP-dependent protein kinases |
| Genestein | 10 | μM | No Effect | Inhibits tyrosine phosphorylation |
| GF10923X | 5 | μM | No Effect | Potent PKC inhibitor |
| Go-6976 | 4 | nM | No Effect | Inhibits PKCα and β1 |
| Go-6983 | 10 | nM | No Effect | Selectively inhibits PKCα, β, γ, δ, ξ |
| H-89 | 100 | nM | Increased 20.49% ± 1.54% | Protein kinase A inhibitor |
| H₂O₂ | 1 | nM | Increased 10.21% ± 3.69% | Oxidizing Agent |
| LY-294002 | 50 | μM | Decreased 70.97% ± 2.58% | Inhibits phosphoinositide 3 (PI3) kinase |
| Lysophosphatidic acid | 1 | μM | No Effect | Rho activator |
| ML-7 | 10 | μM | No Effect | Myosin light chain kinase inhibitor |
| N-acetyl cysteine (NAC) | 5 | mM | No Effect | Antioxidant |
| Phenyl arsine oxide (PAO) | 30 | μM | Decreased 71.5% ± 4.11% | Inhibits tyrosine phosphatases |
| PD-98059 | 10 | μM | Increased 13.69% ± 2.37% | Mitogen activated protein kinase (MAPK) inhibitor |
| Phorbol myristate acetate | 100 | nM | Decreased 102.45% ± 0.58% | Pan PKC activator |
| PP1 | 500 | nM | Decreased 96.79% ± 3.31% | Src Kinase inhibitor |
| Pertussis toxin | 1 | μg/ml | No Effect | Uncouples G proteins |
| Rottlerin | 10 | uM | No Effect | Inhibits PKCδ |
| SB-202190 | 400 | nM | No Effect | Inhibits p38 MAPK |
| Spermine NONOate | 100 | μM | Decreased 109.5% ± 5.41% | NO donor |
| Tetraethyl ammonium (TEA) | 10 | mM | No Effect | Blocks inositol triphosphate kinase induced Ca⁺⁺ release |
| TMB-8 | 1 | mM | Decreased 107.8% ± 2.79% | Inhibits intracellular Ca⁺⁺ mobilization |
| U0126 | 1 | μM | Increased 16.08% ± 3.32% | MEK 1 and 2 inhibitor |
| Wortmannin | 100 | nM | No Effect | PI3 kinase inhibitor |
| Y27632 | 300 | nM | No Effect | Rho-associated kinase inhibitor |

Confluent HMEC monolayers were plated on 48 well plates for adhesion studies, transwells for migration studies, and pretreated with a variety of pharmacological inhibitors and activators for 1 hour. Monolayers were washed three times with HBSS to remove all drug effects on the endothelium. To initiate migration, 100 nM LTB₄ was added to the lower compartment and 500,000 neutrophils were added to the upper compartment and allowed to migrate for 3 hrs. For adhesion assays, LTB₄ was added along with 500,000 neutrophils and allowed to adhere for 1 hr (time of max. adhesion as determined in FIG. 1). In all experiments LTB₄ induced migration was significantly greater than control migration (p<0.001). The data are shown as % change in migration and adhesion as assessed by MPO assay. All values are expressed as means+SE. Data were analyzed using a one-way ANOVA with Bonferroni corrections for multiple comparisons. Probability (P) values of <0.05 were considered significant. ***, P<0.001 vs. LTB4; **, P<0.01 vs. LTB4, P<0.05 vs. LTB4; n=4 for all experiments.

While some of these results including inhibition of intracellular Ca⁺⁺ release, PKA activation, MLCK inhibition, and disruption of microfilaments have previously been investigated in relation to their effects on neutrophil migration, most of the other drugs have not been studied. In fact our most robust novel results on the inhibition of neutrophil migration were in response to endothelial PKC activation. We determined that inhibition of intracellular Ca⁺⁺ release, disruption of microfilaments, exogenous nitric oxide, alterations in tyrosine phosphorylation, PI-3kinase inhibition, activation of PKA, and finally activation of PKC in the endothelium alone reduced PMN TEM.

In addition, because no endothelial cell adhesion molecule has been determined to play a role in Type I (neutrophil-mediated) neutrophil migration, we also screened a number of endothelial adhesion molecules and integrins and determined that CD99, LSP-1, αvβ3, and β1 integrin played no apparent role in mediating neutrophil TEM towards an LTB₄ gradient. However, PECAM-1 slightly, but significantly decreased neutrophil migration towards an LTB₄ gradient (%).

### Example 2

### Type I and II Neutrophil TEM is Attenuated by PKC Activators

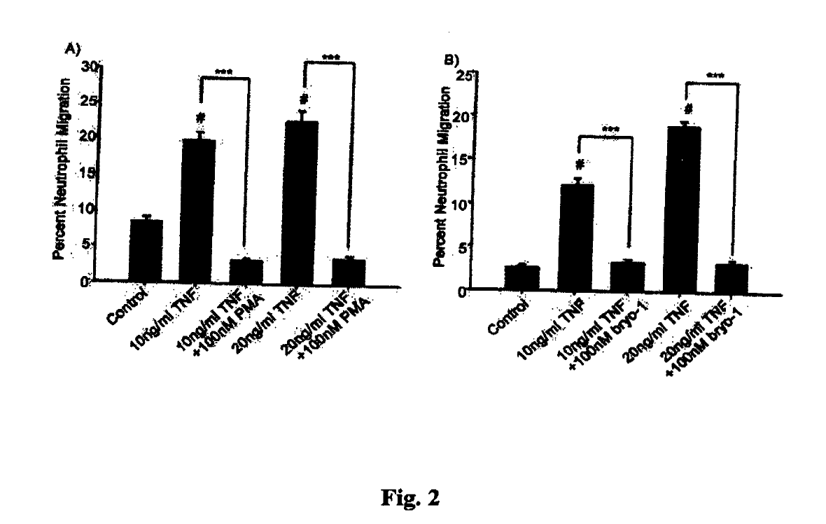

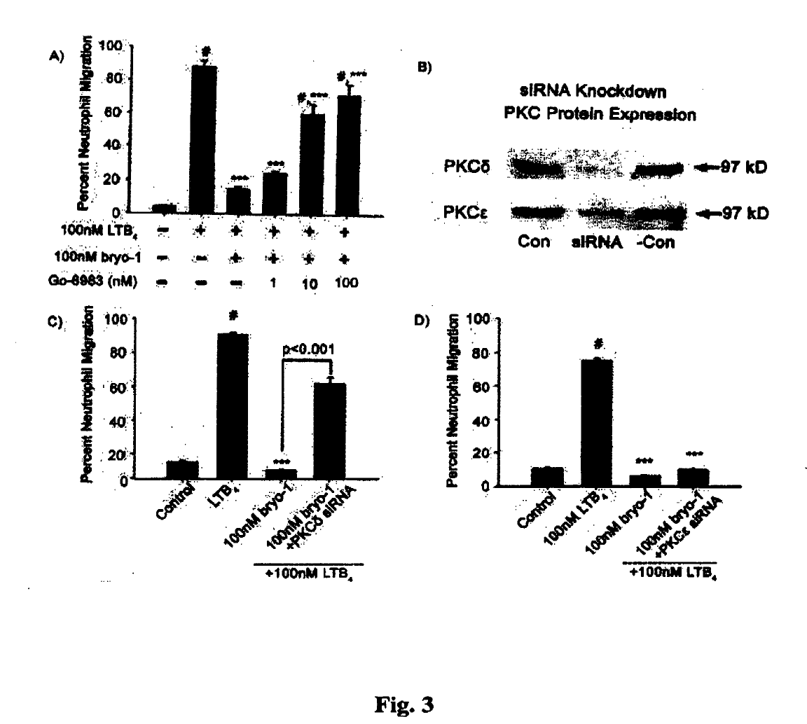

As shown in FIGS. 2 and 3, neutrophil migration was dramatically increased in response to a 100 nM LTB₄ chemoattractant gradient in HMECs.

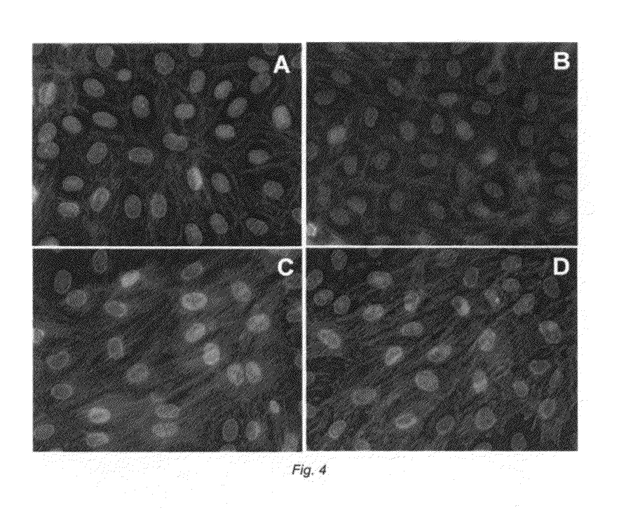

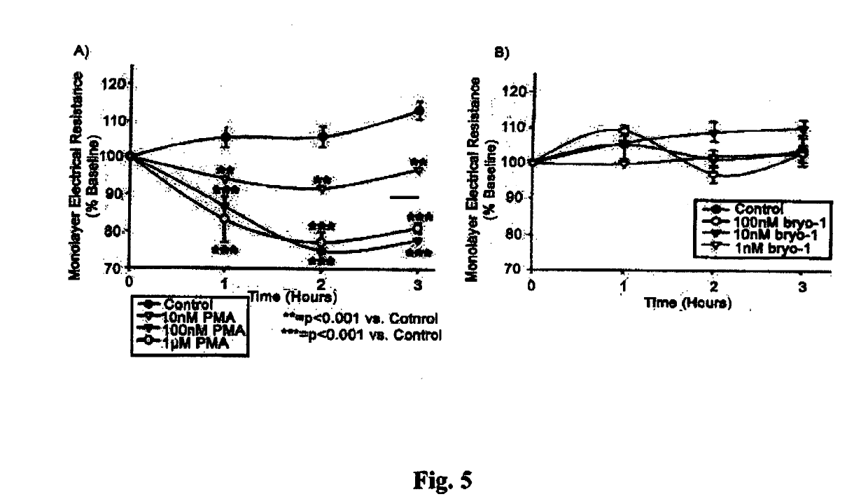

This increase was dose-dependently reduced upon 1 hour PMA or Bryostatin-1 pretreatment (FIGS. 2 and 3). Lower doses of PMA and Bryostatin-1 (1 nM) did not reduce PMN TEM, however, higher doses attenuated neutrophil TEM to levels below that of basal migration (100 nM). Because there are two types of neutrophil migration that are differentially regulated, we wanted to determine if PKC activators could also block neutrophil TEM induced by a cytokine. HMEC TNF-α stimulation for 24 hr induced significant increases in neutrophil migration (FIGS. 4 and 5).

Both PMA (100 nM) and Bryostatin-1 (100 nM) additionally attenuated Type II migration induced by 24 hr treatment with TNF-α (10, 20 ng/ml) (FIGS. 4 and 5). Also, PMA and Bryostatin pretreatments reduced migration induced by a combination of 24 hr TNF-α pretreatment and a 100 nM LTB₄ gradient (data not shown). Neither PMA, nor Bryostatin-1 reduced LTB₄ induced adhesion to the endothelial monolayer, suggesting that the effects of PMA and Bryostatin-1 were directly through alterations in PMN migration (data not shown).

In addition, a single 1 hr 100 nM PMA pretreatment maintained a reduction in LTB₄ induced migration up to 48 hrs in HUVECs (data not shown) and 24 hrs in HMECs, while 1 hr 100 nM Bryostatin pretreatment maintained the reduction in migration for 48 hrs in both cell types (FIGS. 6 and 7). These treatments had no effect on HMEC, or HUVEC viability at any given time point (1, 24, 48 hr) with the exception of a slight but significant reduction in viability of HUVECs 48 hr after a single 1 hr treatment with 100 nM PMA.

### Example 3

### Activation of PKCδ Reduces Neutrophil Migration in Response to LTB₄

To insure that PMA and Bryostatin-1 effects were dependent on PKC activation, we tested various PKC inhibitors in the restoration of PMN TEM in response to LTB₄. While Rottlerin (a PKCδ inhibitor), GF10923X (pan PKC inhibitor more potent for α, βI, βII, γ more potently), GO-6976 (inhibits PKCα and β with no effect on δ, e, ζ) and staurosporine (pan PKC inhibitor) did not restore PMN TEM, Go-6983 dose dependently restored neutrophil migration in response to LTB₄ (FIG. 8). It has been suggested that different concentrations of Go-6983 inhibit different PKC isoforms. Lower concentrations (1 nM) have been suggested to inhibit classical PKC isoforms, while higher concentrations (10 nM) have been suggested to inhibit novel PKC isoforms.

Therefore we choose to investigate the roles of PKCδ and PKCε activation in inhibiting neutrophil TEM. PKCδ and ε siRNA were used to determine each isoforms role in this process. As assessed by FACs for GFP and control nucleofected HUVECs, 2 ug nucleofected GFP resulted in a 60% expression of GFP in HUVECs versus 0.48% in control nucleofected cells. Therefore nucleofection of HUVECs with siRNA should reduce PKC expression in at least 60% of nucleofected cells. PKCδ and ε siRNA nucleofection significantly reduced both PKCδ and ε expression as shown by Western blot (FIG. 9).

Surprisingly, PKCε siRNA nucleofection did not restore LTB₄ induced migration after either PMA or Bryostatin-1 pretreatment suggesting that the effects of PMA and Bryostatin-1 were not through activation of the PKCε isoform (FIG. 10). However, PKCδ siRNA nucleofection restored % of LTB₄ induced neutrophil TEM implicating a role for PKCδ activation in inhibiting LTB₄ induced PMN TEM (FIG. 10). This suggested that PKCδ activation is at least partially responsible for Bryostatin-1 effects on neutrophil migration induced by LTB₄.

What is claimed is:

1. A method of treating or preventing chronic inflammatory disease or transplantation injury, comprising the steps of administering an effective amount of bryostatin-1, bryostatin-1 analog or a pharmaceutically acceptable salt thereof to reduce or prevent induced neutrophil transendothelial migration.

2. The method of claim 1 wherein said effective amount of bryostatin-1, bryostatin analog or pharmaceutically acceptable salt thereof is 10⁻⁷ M per 50 to 90 kg of individual being treated.

3. The method of claim 1 wherein said effective amount of bryostatin-1, bryostatin analog or pharmaceutically acceptable salt thereof is 500 micrograms for 50 to 90 kg of individual being treated.

4. The method of claim 1 wherein said effective amount bryostatin-1, bryostatin-1 analog or a pharmaceutically acceptable salt thereof is held in a dosage form.

5. The method of claim 4 wherein said dosage form is an oral dosage form.

6. The method of claim 5 wherein said oral dosage form is a solid oral dosage form.

7. The method of claim 4 wherein said effective amount of bryostatin-1, bryostatin-1 analog or a pharmaceutically acceptable salt thereof is dispersed or dissolved in a saturated polyalkylene glycol glyceride.

8. The method of claim 7 wherein said polyalkylene glycol glyceride is a mixture of polyalkylene esters of one or more eight carbon to eighteen carbon saturated fatty acids with glycerol.

9. The method of claim 8 wherein said polyalkylene glycol is a polyethylene glycol having a molecular weight of 1000 to 2000.

10. The method of claim 9 wherein said polyalkylene glycol is a polyethylene glycol having a molecular weight of 1400 to 1600.

11. The method of claim 5 wherein said bryostatin-1, bryostatin-1 analog or pharmaceutically acceptable salt thereof is present in said oral dosage form in an amount of 1 to 30% by weight.

12. The method of claim 11 wherein said bryostatin-1, bryostatin-1 analog or pharmaceutically acceptable salt thereof is present in said oral dosage form in an amount of 10 to 20% by weight.

13. The method of claim 11 wherein said bryostatin-1, bryostatin-1 analog or pharmaceutically acceptable salt thereof is present in said oral dosage form in an amount of 2 to 25% by weight.

14. The method of claim 2 wherein said dosage form is a pharmaceutical parenteral formulation.

15. The method of claim 14 wherein said pharmaceutical formulation comprises polyalkylene glycol glyceride.

16. The method of claim 15 wherein said polyalkylene glycol glyceride is a mixture of polyalkylene esters of one or more eight carbon to eighteen carbon saturated fatty acids with glycerol.

17. The method of claim 15 wherein said polyalkylene glycol is a polyethylene glycol having a molecular weight of 1000 to 2000.

18. The method of claim 17 wherein said polyalkylene glycol is a polyethylene glycol having a molecular weight of 1400 to 1600.

19. The method of claim 14 wherein said bryostatin-1, bryostatin-1 analog or pharmaceutically acceptable salt thereof is present in said pharmaceutical formulation in an amount of 1 to 30% by weight.

20. The method of claim 19 wherein said bryostatin-1, bryostatin-1 analog or pharmaceutically acceptable salt thereof is present in said pharmaceutical formulation in an amount of 10 to 20% by weight.

21. The method of claim 19 wherein said bryostatin-1, bryostatin-1 analog or pharmaceutically acceptable salt thereof is present in said pharmaceutical parenteral formulation in an amount of 2 to 25% by weight.

22. The method of claim 14 wherein said −1, bryostatin-1 analog or pharmaceutically acceptable salt thereof is present in said pharmaceutical parenteral formulation as a dispersion in water having a concentration of 0.5 to 70% by weight.

23. The method of claim 1 wherein said chronic inflammatory disease or transplantation injury is in humans.

24. The method of claim 23 wherein said chronic inflammatory disease or transplantation injury is leukocyte mediated tissue injury.

25. The method of claim 23 wherein said chronic inflammatory disease or transplantation injury is mammalian ischemic, transplantation and leukocyte mediated injury and diseases.

26. A dosage form for treating chronic inflammatory disease or transplantation injury comprising an effective amount of bryostatin-1, bryostatin-1 analog or a pharmaceutically acceptable salt thereof to reduce or prevent induced neutrophil transendothelial migration.

27. The dosage form of claim 26 wherein said dosage form is a solid oral dosage form.

28. The dosage form of claim 26 wherein said effective amount of bryostatin-1, bryostatin-1 analog or a pharmaceutically acceptable salt thereof is dispersed or dissolved in a saturated polyalkylene glycol glyceride.

29. The dosage form of claim 28 wherein said polyalkylene glycol glyceride is a mixture of polyalkylene esters of one or more eight carbon to eighteen carbon saturated fatty acids with glycerol.

30. The dosage form of claim 29 wherein said polyalkylene glycol is a polyethylene glycol having a molecular weight of 1000 to 2000.

31. The dosage form of claim 30 wherein said polyalkylene glycol is a polyethylene glycol having a molecular weight of 1400 to 1600.

32. The dosage form of claim 31 wherein said bryostatin-1, bryostatin-1 analog or pharmaceutically acceptable salt thereof is present in said oral dosage form in an amount of 1 to 30% by weight.

33. The dosage form of claim 32 wherein said bryostatin-1, bryostatin-1 analog or pharmaceutically acceptable salt thereof is present in said oral dosage form in an amount of 10 to 20% by weight.

34. The dosage form of claim 27 wherein said bryostatin-1, bryostatin-1 analog or pharmaceutically acceptable salt thereof is present in said oral dosage form in an amount of 2 to 25% by weight.

35. The dosage form of claim 26 wherein said dosage form is a pharmaceutical parenteral formulation.

36. The dosage form of claim 35 wherein said pharmaceutical parenteral formulation comprises polyalkylene glycol glyceride.

37. The dosage form of claim 36 wherein said polyalkylene glycol glyceride is a mixture of polyalkylene esters of one or more eight carbon to eighteen carbon saturated fatty acids with glycerol.

38. The dosage form of claim 37 wherein said polyalkylene glycol is a polyethylene glycol having a molecular weight of 1000 to 2000.

39. The dosage form of claim 38 wherein said polyalkylene glycol is a polyethylene glycol having a molecular weight of 1400 to 1600.

40. The dosage form of claim 26 wherein said bryostatin-1, bryostatin-1 analog or pharmaceutically acceptable salt thereof is present in said pharmaceutical formulation in an amount of 1 to 30% by weight.

41. The dosage form of claim 40 wherein said bryostatin-1, bryostatin-1 analog or pharmaceutically acceptable salt thereof is present in said pharmaceutical formulation in an amount of 10 to 20% by weight.

42. The dosage form of claim 26 wherein said bryostatin-1, bryostatin-1 analog or pharmaceutically acceptable salt thereof is present in said pharmaceutical parenteral formulation in an amount of 2 to 25% by weight.

43. The dosage form of claim 26 wherein said −1, bryostatin-1 analog or pharmaceutically acceptable salt thereof is present in said pharmaceutical parenteral formulation as a dispersion in water having a concentration of 0.5 to 70% by weight.
